(19) United States
(12) Patent Application Publication
(10) Pub. No.: US 2025/0359831 A1
(43) Pub. Date: Nov. 27, 2025

(54) MEDICAL RADIATION SYSTEM ALIGNMENT

(71) Applicant: LEO CANCER CARE, INC., Middleton, WI (US)

(72) Inventors: Stephen Towe, East Sussex (GB); Mark Strangeman, West Sussex (GB); Janusz Harasimowicz, Hertfordshire (GB)

(21) Appl. No.: 18/879,609

(22) PCT Filed: Aug. 17, 2023
(86) PCT No.: PCT/US2023/072346

§ 371 Date: Dec. 27, 2024

Related U.S. Application Data
(60) Provisional application No. 63/399,862, filed on Aug. 22, 2022.

(51) Int. Cl. A61B 6/04; A61B 6/58; A61B 90/00

(52) U.S. Cl. CPC A61B 6/0407; A61B 6/584; A61B 90/39; A61B 2090/3966

(57) ABSTRACT

Provided herein is technology relating to medical radiation systems and particularly, but not exclusively, to apparatuses, methods, and systems for aligning a radiation source with a patient positioning system and/or patient rotation system for use in medical diagnostic imaging and/or radiotherapy.

This application claims priority to U.S. provisional patent application Ser. No. 63/399,862, filed Aug. 22, 2022, which is incorporated herein by reference in its entirety.

### FIELD

Provided herein is technology relating to medical radiation systems and particularly, but not exclusively, to apparatuses, methods, and systems for aligning a radiation source with a patient positioning system and/or patient rotation system for use in medical diagnostic imaging and/or radiotherapy.

### BACKGROUND

Medical radiation systems employ radiation sources for imaging and therapeutic purposes, e.g., for computed tomographic imaging and in radiotherapy. Medical therapy and imaging procedures typically involve immobilizing a patient on a bed or a chair and moving a radiation source around the patient to target the relevant area of the body of the patient. The technologies used for such procedures often comprise complex mechanisms to provide stability for the medical radiation systems and extensive shielding for the moving radiation source. Thus, conventional medical radiation systems are generally expensive to install and maintain.

U.S. Pat. App. Pub. No. 20200268327 and U.S. Pat. App. Ser. No. 63/237,513, each of which is incorporated herein by reference, describe patient positioning assemblies and patient positioning systems for orienting a patient with respect to a static radiation source and for moving, positioning, and/or rotating the body of the patient as needed for imaging or treatment. Alignment of the patient positioning assemblies and patient positioning systems with the radiation source may improve treatment and imaging of patients. Thus, use of medical radiation systems would benefit from technologies relating to aligning a medical radiation system with a patient positioning assembly or and patient positioning system.

### SUMMARY

Provided herein is technology relating to medical radiation systems and particularly, but not exclusively, to apparatuses, methods, and systems for aligning a radiation source with a patient positioning system and/or patient rotation system for use in medical diagnostic imaging and/or radiotherapy.

In particular, the technology provided herein relates to medical diagnostic (e.g., imaging) and/or therapeutic radiological technologies in which a patient support assembly (e.g., comprising a patient) rotates around an axis as described herein. The technology provides for adjusting the axis of rotation of the patient support assembly (e.g., comprising a patient) to align an imaging beam and/or a treatment beam with respect to any point in the patient by computing compensatory translations of the patient support assembly and/or of a patient along one or more of three translation axes.

Furthermore, in some embodiments, the technology provides for adjusting any axis of rotation of the patient support assembly (e.g., comprising a patient) to align an imaging and/or treatment beam with respect to any point in the patient by computing compensatory translations of the patient along one or more translation axes. In particular, embodiments provide methods in which a first step comprises calculating the compensatory translations along one or more axes; and a second step comprises adding the independent translations vectorally.

Accordingly, in some embodiments, the technology provides a method of aligning a medical radiation system comprising a patient rotation system and a radiation source. For example, in some embodiments, methods comprise rotating the patient rotation system about a rotation axis; detecting a radiation beam from the radiation source to produce images of a reference target located on a patient support assembly of the patient rotation system; analyzing the images to determine a displacement of the patient support assembly relative to the rotation axis; and adjusting the patient support assembly to align the patient support assembly relative to the rotation axis. In some embodiments, the displacement is determined by comparing a location and an orientation of the reference target relative to the rotation axis. In some embodiments, adjusting the patient support assembly comprises aligning a center of rotation of the reference target with the rotation axis. In some embodiments, methods further comprise analyzing the images to locate a central axis of the radiation beam relative to the rotation axis; and adjusting the radiation source such that the central axis of the radiation beam intersects the rotation axis. In some embodiments, the patient rotation system comprises a base configured to rotate. In some embodiments, the base supports the patient support assembly. In some embodiments, the patient support assembly is configured for movement with six degrees of freedom relative to the base. In some embodiments, adjusting the patient support assembly comprises effectuating a translational movement of the patient support assembly relative to the base. In some embodiments, adjusting the patient support assembly comprises effectuating a rotational movement of the patient support assembly relative to the base. In some embodiments, rotating the patient rotation system comprises rotating the base and the rotation axis is an axis of the base. In some embodiments, the rotation axis is an axis of symmetry of the base. In some embodiments, the medical radiation system further comprises an imaging device for detecting the radiation beam and producing the images of the reference target. In some embodiments, the imaging device is located opposite the radiation source relative to the patient support assembly. In some embodiments, the images of the reference target comprise at least two images of the reference target produced for different angles of rotation of the patient rotation system.

In some embodiments, the reference target comprises a body and one or more markers fixed to the body in a prearranged configuration. In some embodiments, the one or more markers are fixed to the body in a prearranged configuration such that each marker is at least partly exposed to the radiation beam at each angle of rotation of the patient rotation system. In some embodiments, the one or more markers are disposed on an imaginary plane in the body, and wherein, when the reference target is located on the patient support assembly, the imaginary plane is tilted relative to the rotation axis. In some embodiments, the reference target comprises a central marker disposed in the body at the center of rotation of the reference target. In some embodiments, the body is transparent (e.g., radiolucent) to the radiation beam. In some embodiments, the one or more markers are opaque (e.g., radiopaque) to the radiation beam. In some embodiments, the radiation source is an imaging radiation source or a therapeutic radiation source.

In some embodiments, the radiation source is a first radiation source (e.g., producing a first radiation beam) and the medical radiation system further comprises a second radiation source (e.g., producing a second radiation beam). In some embodiments, methods further comprise detecting a second radiation beam from the second radiation source to produce additional images of the reference target; analyzing the additional images to locate a central axis of the second radiation beam relative to the rotation axis; and adjusting the second radiation source such that the central axis of the second radiation beam intersects the rotation axis. In some embodiments, methods further comprise adjusting at least one of the first radiation source and/or the second radiation source such that the central axis of the second radiation beam intersects the central axis of the first radiation beam.

In some embodiments, methods further comprise attaching the reference target to the patient support assembly. In some embodiments, the patient support assembly comprises an interface for attaching the reference target at a fixed position on the patient support assembly.

In some embodiments, the technology provides systems. For example, in some embodiments, a system comprises a medical radiation system; and a reference target comprising a body and one or more markers fixed to the body in a prearranged configuration. In some embodiments, systems comprise a detector. In some embodiments, systems comprise a patient support assembly. In some embodiments, the patient support assembly comprises an interface structured to accept said reference target. In some embodiments, the system is structured to rotate said reference target around an axis orthogonal to an axis between a source and a detector. In some embodiments, the medical radiation system comprises a static source. In some embodiments, systems comprise a software component comprising instructions for rotating a patient rotation system about a rotation axis; controlling a radiation beam; receiving a signal and/or data from a detector for producing images: analyzing the images to determine a displacement of the patient support assembly relative to the rotation axis; analyzing images to locate an isocenter of a radiation beam relative to the rotation axis; and/or determining a displacement of the central axis of a radiation beam relative to the rotation axis. In some embodiments, systems comprise a component structured to adjust the patient support assembly to align the patient support assembly relative to a rotation axis of the patient support assembly. In some embodiments, systems comprise a component structured to adjust the position of a radiation source and/or a position of a beam produced by said radiation source.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings.

FIG. 1A is a schematic drawing showing a perspective view of an example medical radiation system.

FIG. 1B is a schematic drawing showing a top view of a medical radiation system.

FIG. 1C is a schematic drawing showing a top view of a medical radiation system. A patient is positioned in the path of a beam.

FIG. 1D is a schematic drawing showing a top view of a medical radiation system comprising two sources. A patient is positioned in the path of a beam produced by one of the two sources.

FIG. 1E is a schematic drawing showing a top view of a medical radiation system comprising two sources. A patient is positioned in the path of a beam produced by one of the two sources.

FIG. 1F is a schematic drawing showing a top view of a medical radiation system. A reference target is positioned in the path of a beam.

FIG. 2 is a block diagram of an example method of aligning a medical radiation system.

FIG. 3 is a drawing showing a perspective view of an example reference target for use with the method of FIG. 2.

FIG. 4 is a drawing of a top view of the reference target of FIG. 3.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to medical radiation systems and particularly, but not exclusively, to apparatuses, methods, and systems for aligning a radiation source with a patient positioning system and/or patient rotation system for use in medical diagnostic imaging and/or radiotherapy. In some embodiments, the technology provides embodiments of a method for aligning a medical radiation system comprising a patient rotation system and a radiation source. In some embodiments, methods comprise rotating the patient rotation system about a rotation axis, detecting a radiation beam from the radiation source, and producing images of a reference target located on a patient support assembly of the patient rotation system. In some embodiments, methods further comprise analyzing the images to determine a displacement of the patient support assembly relative to the rotation axis and adjusting the patient support assembly to align the patient support assembly relative to the rotation axis. In some embodiments, methods further comprise analyzing the images to determine a displacement of a central axis of the radiation beam relative to the rotation axis and adjusting the radiation source such that the central axis of the radiation beam intersects the rotation axis.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

As used herein, the term “computed tomography” is abbreviated “CT” and refers both to tomographic and non-tomographic radiography. For instance, the term “CT” refers to numerous forms of CT, including but not limited to x-ray CT, positron emission tomography (PET), single-photon emission computed tomography (SPECT), and photon counting computed tomography. Generally, computed tomography (CT) comprises use of an x-ray source and a detector that rotates around a patient and subsequent reconstruction of images into different planes. In embodiments of CT (e.g., devices, apparatuses, and methods provided for CT) described herein, the x-ray source is a static source and the patient is rotated with respect to the static source. Currents for x-rays used in CT describe the current flow from a cathode to an anode and are typically measured in milliamperes (mA).

As used herein, the term “structured to [verb]” means that the identified element or assembly has a structure that is shaped, sized, disposed, coupled, and/or configured to perform the identified verb. For example, a member that is “structured to move” is movably coupled to another element and includes elements that cause the member to move or the member is otherwise configured to move in response to other elements or assemblies. As such, as used herein, “structured to [verb]” recites structure and not function. Further, as used herein, “structured to [verb]” means that the identified element or assembly is intended to, and is designed to, perform the identified verb. As used herein, the term “associated” means that the elements are part of the same assembly and/or operate together or act upon/with each other in some manner. For example, an automobile has four tires and four hub caps. While all the elements are coupled as part of the automobile, it is understood that each hubcap is “associated” with a specific tire.

As used herein, the term “coupled” refers to two or more components that are secured, by any suitable means, together. Accordingly, in some embodiments, the statement that two or more parts or components are “coupled” shall mean that the parts are joined or operate together either directly or indirectly, e.g., through one or more intermediate parts or components. As used herein, “directly coupled” means that two elements are directly in contact with each other. As used herein, “fixedly coupled” or “fixed” means that two components are coupled so as to move as one while maintaining a constant orientation relative to each other. Accordingly, when two elements are coupled, all portions of those elements are coupled. A description, however, of a specific portion of a first element being coupled to a second element, e.g., an axle first end being coupled to a first wheel, means that the specific portion of the first element is disposed closer to the second element than the other portions thereof. Further, an object resting on another object held in place only by gravity is not “coupled” to the lower object unless the upper object is otherwise maintained substantially in place. That is, for example, a book on a table is not coupled thereto, but a book glued to a table is coupled thereto.

As used herein, the term “removably coupled” or “temporarily coupled” means that one component is coupled with another component in an essentially temporary manner. That is, the two components are coupled in such a way that the joining or separation of the components is easy and does not damage the components. Accordingly, “removably coupled” components is readily uncoupled and recoupled without damage to the components.

As used herein, the term “operatively coupled” means that a number of elements or assemblies, each of which is movable between a first position and a second position, or a first configuration and a second configuration, are coupled so that as the first element moves from one position/configuration to the other, the second element moves between positions/configurations as well. It is noted that a first element is “operatively coupled” to another without the opposite being true.

As used herein, the term “rotatably coupled” refers to two or more components that are coupled in a manner such that at least one of the components is rotatable with respect to the other.

As used herein, the term “translatably coupled” refers to two or more components that are coupled in a manner such that at least one of the components is translatable with respect to the other.

As used herein, the term “temporarily disposed” means that a first element or assembly is resting on a second element or assembly in a manner that allows the first element/assembly to be moved without having to decouple or otherwise manipulate the first element. For example, a book simply resting on a table, e.g., the book is not glued or fastened to the table, is “temporarily disposed” on the table.

As used herein, the term “correspond” indicates that two structural components are sized and shaped to be similar to each other and is coupled with a minimum amount of friction. Thus, an opening which “corresponds” to a member is sized slightly larger than the member so that the member may pass through the opening with a minimum amount of friction. This definition is modified if the two components are to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening. With regard to surfaces, shapes, and lines, two, or more, “corresponding” surfaces, shapes, or lines have generally the same size, shape, and contours.

As used herein, a “path of travel” or “path,” when used in association with an element that moves, includes the space an element moves through when in motion. As such, any element that moves inherently has a “path of travel” or “path.”

As used herein, the statement that two or more parts or components “engage” one another shall mean that the elements exert a force or bias against one another either directly or through one or more intermediate elements or components. Further, as used herein with regard to moving parts, a moving part may “engage” another element during the motion from one position to another and/or may “engage” another element once in the described position. Thus, it is understood that the statements, “when element A moves to element A first position, element A engages element B,” and “when element A is in element A first position, element A engages element B” are equivalent statements and mean that element A either engages element B while moving to element A first position and/or element A either engages element B while in element A first position.

As used herein, the term “operatively engage” means “engage and move.” That is, “operatively engage” when used in relation to a first component that is structured to move a movable or rotatable second component means that the first component applies a force sufficient to cause the second component to move. For example, a screwdriver is placed into contact with a screw. When no force is applied to the screwdriver, the screwdriver is merely “coupled” to the screw. If an axial force is applied to the screwdriver, the screwdriver is pressed against the screw and “engages” the screw. However, when a rotational force is applied to the screwdriver, the screwdriver “operatively engages” the screw and causes the screw to rotate. Further, with electronic components, “operatively engage” means that one component controls another component by a control signal or current.

As used herein, the term “number” shall mean one or an integer greater than one (e.g., a plurality).

As used herein, in the phrase “[x] moves between its first position and second position,” or, “[y] is structured to move [x] between its first position and second position,” “[x]” is the name of an element or assembly. Further, when [x] is an element or assembly that moves between a number of positions, the pronoun “its” means “[x],” i.e., the named element or assembly that precedes the pronoun “its.”

As used herein, a “radial side/surface” for a circular or cylindrical body is a side/surface that extends about, or encircles, the center thereof or a height line passing through the center thereof. As used herein, an “axial side/surface” for a circular or cylindrical body is a side that extends in a plane extending generally perpendicular to a height line passing through the center. That is, generally, for a cylindrical soup can, the “radial side/surface” is the generally circular sidewall and the “axial side(s)/surface(s)” are the top and bottom of the soup can.

As used herein, a “diagnostic” test includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject having a cancer or the likelihood that such a subject will respond favorably to a compound (e.g., a pharmaceutical, e.g., a drug) or other treatment.

As used herein, the term “condition” refers generally to a disease, malady, injury, event, or change in health status.

As used herein, the term “treating” or “treatment” with respect to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, “treatment” comprises exposing a patient or a portion thereof (e.g., a tissue, organ, body part, or other localize region of a patient body) to radiation (e.g., electromagnetic radiation, ionizing radiation).

As used herein, the term “beam” refers to a stream of radiation (e.g., electromagnetic wave and/or or particle radiation). In some embodiments, the beam is produced by a source and is restricted to a small-solid angle. In some embodiments, the beam is collimated. In some embodiments, the beam is generally unidirectional. In some embodiments, the beam is divergent.

As used herein, the term “patient” or “subject” refers to a mammalian animal that is identified and/or selected for imaging and/or treatment with radiation.

Accordingly, in some embodiments, a patient or subject is contacted with a beam of radiation, e.g., a primary beam produced by a radiation source. In some embodiments, the patient or subject is a human. In some embodiments, the patient or subject is a veterinary or farm animal, a domestic animal or pet, or animal used for clinical research. In some embodiments, the subject or patient has cancer and/or the subject or patient has either been recognized as having or at risk of having cancer.

As used herein, the term “treatment volume” or “imaging volume” refers to the volume (e.g., tissue) of a patient that is selected for imaging and/or treatment with radiation. For example, in some embodiments, the “treatment volume” or “imaging volume” comprises a tumor in a cancer patient. As used herein, the term “healthy tissue” refers to the volume (e.g., tissue) of a patient that is not and/or does not comprise the treatment volume. In some embodiments, the imaging volume is larger than the treatment volume and comprises the treatment volume.

As used herein, the term “radiation source” or “source” refers to an apparatus that produces radiation (e.g., ionizing radiation) in the form of photons (e.g., described as particles or waves). In some embodiments, a radiation source is a linear accelerator (“linac”) that produces x-rays or electrons to treat a cancer patient by contacting a tumor with the x-ray or electron beam. In some embodiments, the source produces particles (e.g., photons, electrons, neutrons, hadrons, ions (e.g., protons, carbon ions, other heavy ions)). In some embodiments, the source produces electromagnetic waves (e.g., x-rays and gamma rays having a wavelength in the range of approximately 1 pm to approximately 1 nm). While it is understood that radiation can be described as having both wave-like and particle-like aspects, it is sometimes convenient to refer to radiation in terms of waves and sometimes convenient to refer to radiation in terms of particles. Accordingly, both descriptions are used throughout without limiting the technology and with an understanding that the laws of quantum mechanics provide that every particle or quantum entity is described as either a particle or a wave.

As used herein, the term “static source” refers to a source that does not revolve around a patient during use of the source for imaging or therapy. In particular, a “static source” remains fixed with respect to an axis passing through the patient while the patient is being imaged or treated. While the patient may rotate around said axis to produce relative motion between the static source and rotating patient that is equivalent to the relative motion of a source revolving around a static patient, a static source does not move with reference to a third object, frame of reference (e.g., a treatment room in which a patient is positioned), or patient axis of rotation during imaging or treatment, while the patient is rotated with respect to said third object, said frame of reference (e.g., said treatment room in which said patient is positioned), or patient axis of rotation through the patient during imaging or treatment. Thus, a static source is installed on a mobile platform and thus the static source may move with respect to the Earth and fixtures on the Earth as the mobile platform moves to transport the static source. Thus, the term “static source” may refer to a mobile “static source” provided that the mobile “static source” does not revolve around an axis of rotation through the patient during imaging or treatment of the patient. Further, the static source may translate and/or revolve around the patient to position the static source prior to imaging or treatment of the patient or after imaging or treatment of the patient. Thus, the term “static source” may refer to a source that translates or revolves around the patient in non-imaging and non-treatment use, e.g., to position the source relative to the patient when the patient is not being imaged and/or treated. In some embodiments, the “static source” is a photon source and thus is referred to as a “static photon source”.

As used herein, the term “detector” refers to a sensor for detecting photons, e.g., as produced by a source (e.g., a static source). Accordingly, embodiments provide a detector for imaging and/or for alignment of a patient support. In some embodiments, a detector is an electromagnetic radiation detector, an X-ray detector, a photon detector, and/or a gamma ray detector. Thus, in some embodiments, a detector may detect photons of visible light. Embodiments provide a detector for CT, PET, SPECT, photon counting computed tomography, and/or portal imaging.

A detector may comprise a plurality of “detector elements”, each of which comprises an array of pixels. As used herein, the term “pixel”, when referring to a detector, refers to the smallest discrete element of photon sensing by a photon detector element and thus also by a detector. In some embodiments, a pixel has an area of approximately 1 mm×1 mm. A detector element comprises a component for detecting photons at each pixel and a component for outputting an electrical signal (e.g., a current or voltage) for each pixel corresponding to photons detected by each pixel of the detector element. An integrating (“indirect”) detector element comprises a scintillator and a photodiode (e.g., a semiconductor). Photons impacting the scintillator produce a number of visible light photons that are absorbed by the photodiode. The photodiode measures the amount of light produced by the scintillator and generates an electrical signal (e.g., a current or voltage) proportional to the total energy deposited during a measurement interval. A photon counting (“direct”) detector element comprises a photodiode (e.g., a semiconductor) that produces a current or voltage for each photon detected by the photodiode (a photon counting detector element does not comprise a scintillator).

Photons absorbed in the semiconductor generate pairs of positive and negative charges that move away from each other due to a voltage applied to the semiconductor. The moving charges generate an electrical signal that is provided to an electronic readout circuit. For example, a photon counting detector element may comprise a multi-channel analyzer that identifies photons based on photon energy and categorizes photons into energy channels based on photon energy. Thus, a photon counting detector element may be used to produce a photon energy spectrum.

As used herein, the term “module” or “detector module” refers to a discrete component of a detector comprising a plurality of detector elements. Accordingly, a number of detector modules may be assembled to provide a detector having a desired design and number of detector elements, e.g., in some embodiments arranged in a number of rows and a number of columns.

Thus, a detector may comprise a number of detector modules, a detector module may comprise a number of detector elements, and a detector element may comprise a number of pixels.

As used herein, the term “Z” refers to an atomic number (e.g., of an element and/or of a material comprising an element). As used herein, the “Z” of a material refers to the atomic number of the element or elements from which the material is made.

As used herein, the term “effective atomic number” or “Z_{eff}” refers to the effective or average atomic number for a compound or mixture of materials (e.g., an alloy). The Z_{eff} may be determined experimentally or estimated according to calculations described by Murty (1965) “Effective Atomic Numbers of Heterogeneous Materials” Nature 207 (4995): 398-99; Taylor (2008) “The effective atomic number of dosimetric gels” Australasian Physics & Engineering Sciences in Medicine 31 (2): 131-38; Taylor (2009) “Electron Interaction with Gel Dosimeters: Effective Atomic Numbers for Collisional, Radiative and Total Interaction Processes” Radiation Research 171 (1): 123-26; Taylor (2011) “Robust determination of effective atomic numbers for electron interactions with TLD-100 and TLD-100H thermoluminescent dosimeters” Nuclear Instruments and Methods in Physics Research Section B: Beam Interactions with Materials and Atoms 269 (8): 770-73; and Taylor (2012) “Robust calculation of effective atomic numbers: The Auto-Z_{eff} software” Medical Physics 39 (4): 1769-78, each of which is incorporated herein by reference. The Auto-Z_{eff} software described by Taylor is freely available for use in calculating the Z_{eff} of compounds or mixtures of materials.

As used herein, the term “attenuation coefficient” or “linear attenuation coefficient” refers to a measure of the extent to which the radiant flux of a beam is reduced as it passes through a specific material, e.g., as a result of absorption and/or scattering. A “mass attenuation coefficient” of a material may be used in which the attenuation coefficient is normalized per unit density of the material, thus providing a value that is constant for a given element or compound.

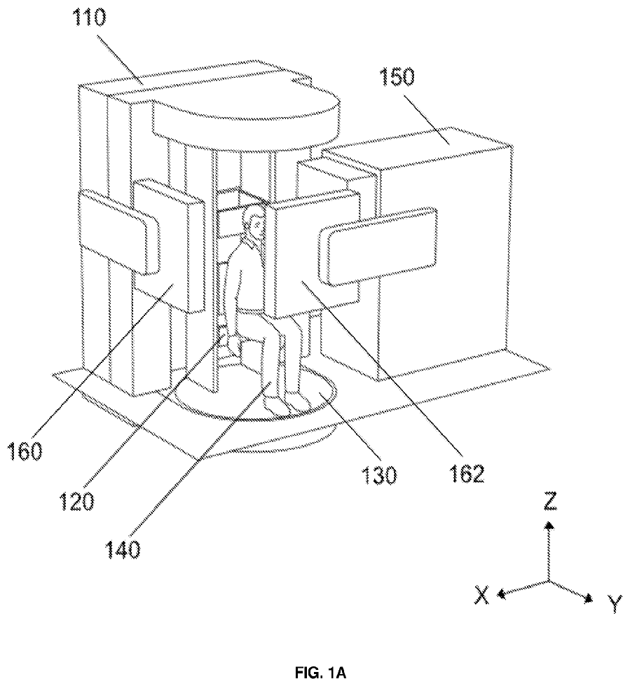

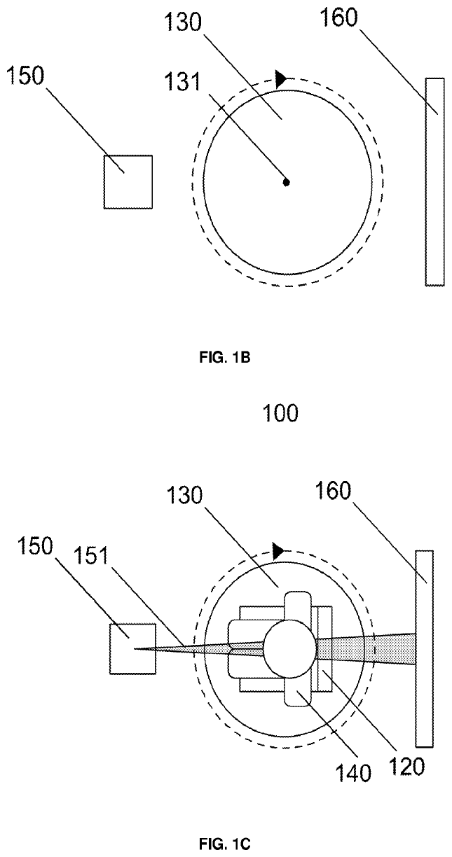

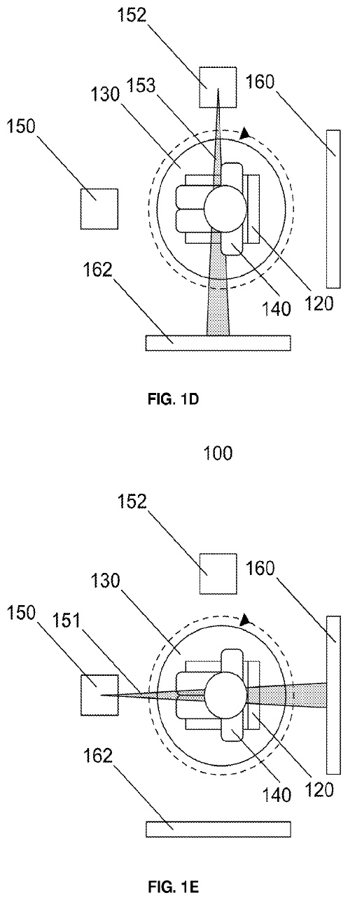

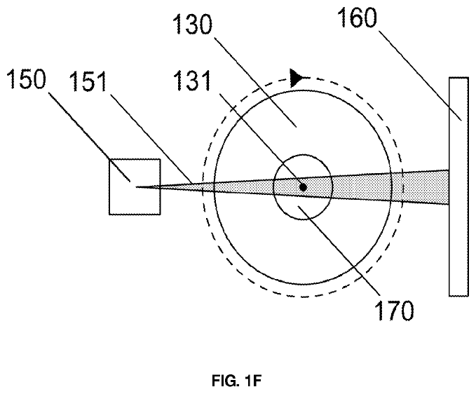

Embodiments of the technology described herein relate to translations along axes and/or rotations around axes. In some embodiments, a coordinate system is used that comprises an X axis, a Y axis, and a Z axis defined with respect to a patient support and/or a patient. See FIG. 1A. As shown in FIG. 1A, embodiments use a coordinate system in which the X axis and Y axis together are in and/or define a horizontal plane and the Z axis is and/or defines a vertical axis. With respect to a patient positioned on the patient support (e.g., the patient positioning apparatus), the X axis is a left-right, horizontal, or frontal axis; the Y axis is an anteroposterior, dorsoventral, or sagittal axis; and the Z axis is a sagittal or longitudinal axis. The X axis and the Y axis together are in and/or define a horizontal, transverse, and/or axial plane. The Y axis and the Z axis together are in and/or define a sagittal or longitudinal plane. The X axis and the Z axis together are in and/or define a frontal or coronal plane.

Accordingly, in some embodiments, descriptions of movements as “forward” or “backward” are movements along the Y axis; descriptions of movements as “left” or “right” are movements along the X axis; and descriptions of movements as “up” and “down” are movements along the Z axis. Furthermore, a rotation described as “roll” is rotation around the Y axis; a rotation described as “pitch” is rotation around the X axis; and a rotation described as “yaw” is rotation around the Z axis. Thus, in some embodiments, technologies are described as having six degrees of freedom, e.g., translations along one or more of the X, Y, and/or Z axes; and/or rotations around one or more of the X, Y, and/or Z axes.

### Medical Radiation System

In some embodiments, the technology relates to aligning components of a medical radiation system. In some embodiments, e.g., as shown in FIGS. 1A to 1F, the technology relates to a medical radiation system 100. In some embodiments, medical radiation system 100 comprises a patient rotation system 110 structured to rotate about a rotation axis. In some embodiments, the patient rotation system 110 comprises a patient positioning system (e.g., comprising a patient positioning apparatus or a configurable patient support assembly 120) mounted onto a base 130. Base 130 is structured to rotate about an axis 131 of base 130. In some embodiments, base 130 is structured to rotate about a vertical axis of symmetry of base 130. In some embodiments, the patient positioning system, patient positioning apparatus, and/or configurable patient support 120 is/are as described in U.S. Pat. App. Pub. No. 20200268327 and U.S. Pat. App. Ser. No. 63/237,513, each of which is incorporated herein by reference.

The patient support assembly 120 is structured to support a patient 140 in an upright (e.g., standing, sitting, perched) position during radiation treatment or imaging. Accordingly, embodiments provide that the configurable patient support assembly 120 is adjustable to support patient 140 in an upright (e.g., standing, sitting, perched) position or any other position in which the torso of patient 140 is in a generally vertical or upright position (e.g., a semi-standing position, a crouching position). In some embodiments, the patient support assembly 120 comprises supporting members such as a seat, a backrest, a headrest, arm rests, shin rests, foot brace (e.g., heel stop), and/or a footrest to assist in supporting and/or immobilizing patient 140 in a given position. In some embodiments, e.g., as described in U.S. Pat. App. Ser. No. 63/237,513, which is incorporated herein by reference, the configurable patient support assembly 120 comprises one or more configurable and movable components, e.g., a back rest (e.g., a configurable and movable back rest), a head rest (e.g., a configurable and movable head rest), an arm rest (e.g., a configurable and movable arm rest), a seat member (e.g., a configurable and movable seat member), a shin rest (e.g., a configurable and movable shin rest), and/or a foot brace (e.g., a configurable and movable foot brace). In some embodiments, one or more configurable and movable components of the configurable patient support comprises one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat member (e.g., a seat member operatively engaged with a seat member motor), a motorized shin rest (e.g., a shin rest operatively engaged with a shin rest motor), and/or a motorized foot brace (e.g., a foot brace operatively engaged with a foot brace motor). In some embodiments, the back rest motor is structured to move (e.g., translate and/or rotate) the back rest, the head rest motor is structured to move (e.g., translate and/or rotate) the head rest, the arm rest motor is structured to move (e.g., translate and/or rotate) the arm rest, the seat member motor is structured to move (e.g., translate and/or rotate) the seam member, the shin rest motor is structured to move (e.g., translate and/or rotate) the shin rest, and/or the foot brace motor is structured to move (e.g., translate and/or rotate) the foot brace. In some embodiments, the technology provides a configurable patient support 120 that is configured in a static configuration. In some embodiments, the technology provides a configurable patient support 120 that is configured in a dynamic configuration (e.g., a configuration that moves to assist patient movement, e.g., for patient ingress and/or for patient egress). See U.S. Pat. App. Ser. No. 63/237,513, which is incorporated herein by reference.

In some embodiments, the patient support assembly 120 is operatively coupled to base 130 such that the patient support assembly 120 rotates together with base 130 (e.g., around axis 131). However, patient support assembly 120 may be adjustably mounted to base 130 for adjusting of a position and/or orientation of patient support assembly 120 relative to base 130. In some embodiments, patient support assembly 120 is configured for movement with six degrees of freedom, permitting translation along three perpendicular axes (e.g., two axes in a horizontal plane and a vertical axis; see FIG. 1A) and rotation about three perpendicular axes (e.g., yaw, pitch, and roll; see FIG. 1A). See U.S. Pat. App. Ser. No. 63/237,513, which is incorporated herein by reference. In some embodiments, patient support assembly 120 is moveable with fewer than six degrees of freedom.

In some embodiments, the medical radiation system 100 comprises a first radiation source 150 structured to generate a beam 151 of electromagnetic radiation. In some embodiments, the first radiation source 150 is a kilovoltage (kV) or a megavoltage (MV) x-ray radiation source. First radiation source 150 may be a therapeutic radiation source or an imaging radiation source. In some embodiments, the medical radiation system further comprises a second radiation source 152 structured to generate a second beam 153 of electromagnetic radiation. Thus, in some embodiments, medical radiation system 100 comprises two radiation sources, e.g., a first radiation source that is a therapeutic radiation source and a second radiation source that is an imaging radiation source. In some embodiments, radiation source 150 is a static source, e.g., a source that is not moveable during normal operation (e.g., during radiotherapy). Accordingly, the radiation source 150 may translate, revolve, and/or rotate during a calibration or alignment procedure. In some embodiments, first radiation source 150 is a static source and/or second radiation source 152 is a static source. Accordingly, the first radiation source 150 and/or the second radiation source 152 may translate, revolve, and/or rotate during a calibration or alignment procedure.

Furthermore, embodiments provide that the radiation beam 151 from the first radiation source 150 is perpendicular to the axis of rotation 131 of base 130 (e.g., following the alignment procedure) and/or that the radiation beam 153 from the second radiation source 152 is perpendicular to the axis 131 of rotation of base 130 (e.g., following the alignment procedure). In some embodiments, the radiation source 150 is oriented such that the radiation beam 151 intersects the axis of rotation 131. In some embodiments, an isocenter of the radiation beam intersects the axis of rotation 131.

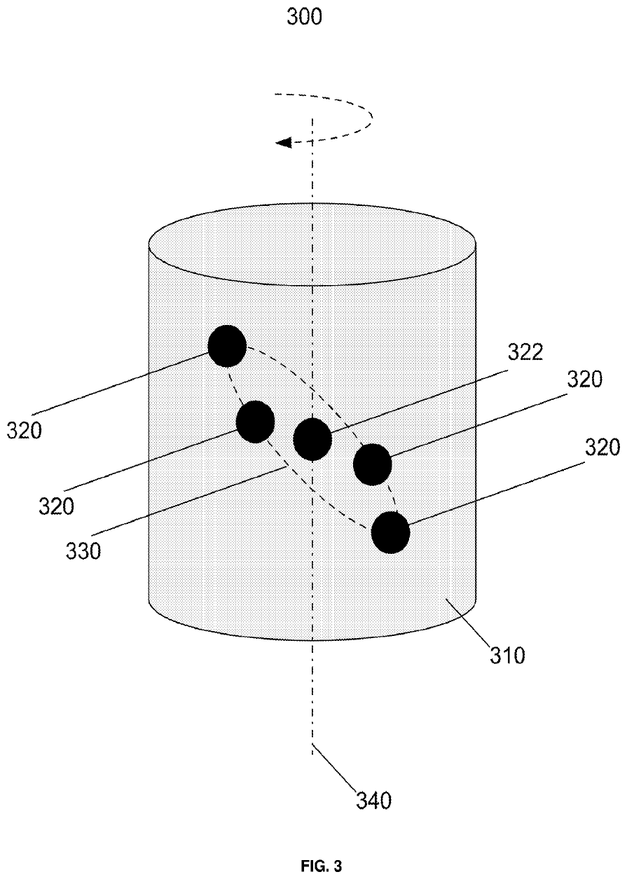

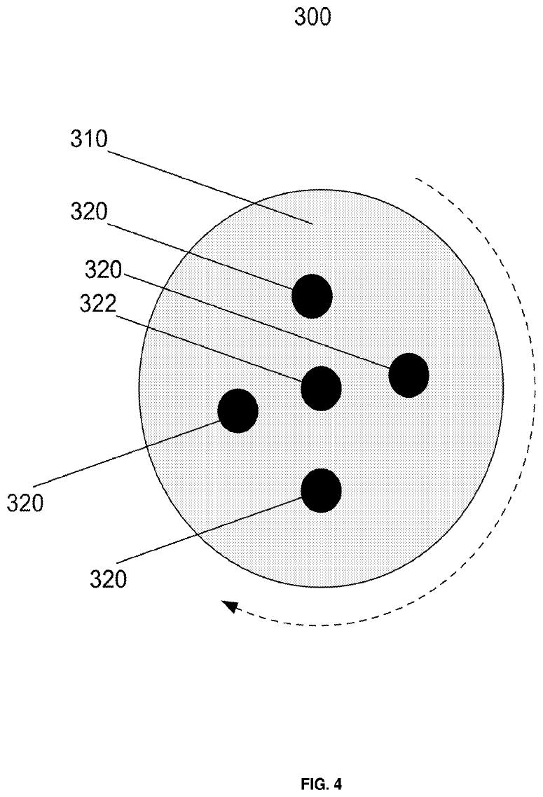

In some embodiments, radiation source 150 is structured to direct a radiation beam 151 in the direction of patient support assembly 120. Accordingly, when a patient 140 is positioned on the patient support assembly 120, radiation source 150 is structured to direct a radiation beam 151 in the direction of patient 140. In some embodiments, the medical radiation system 100 comprises a detector 160 (e.g., a detection panel) provided opposite the radiation source 150 to detect the radiation beam 151 that traverses patient 140. In some embodiments, detector 160 is an imaging device that produces a signal and/or data for generating an image produced by the radiation beam 151. In some embodiments, an additional (e.g., second) detector 162 is associated with the second radiation source 152 of medical radiation system 100. In some embodiments, e.g., as described further herein, a reference target 170 (e.g., a reference target 170 comprising a body and a plurality of markers fixed to the body (e.g., as shown in FIGS. 3 and 4)) is placed between the radiation source 150 and the detector 160 (e.g., a detection panel). In some embodiments, e.g., as described further herein, a reference target 170 (e.g., a reference target 170 comprising a body and a plurality of markers fixed to the body (e.g., as shown in FIGS. 3 and 4)) is placed between the second radiation source 152 and the second detector 162 (e.g., a detection panel). In some embodiments, the technology provides a system comprising a medical radiation system 100 and a reference target 170 (e.g., a reference target 170 comprising a body and a plurality of markers fixed to the body (e.g., as shown in FIGS. 3 and 4)).

### Methods

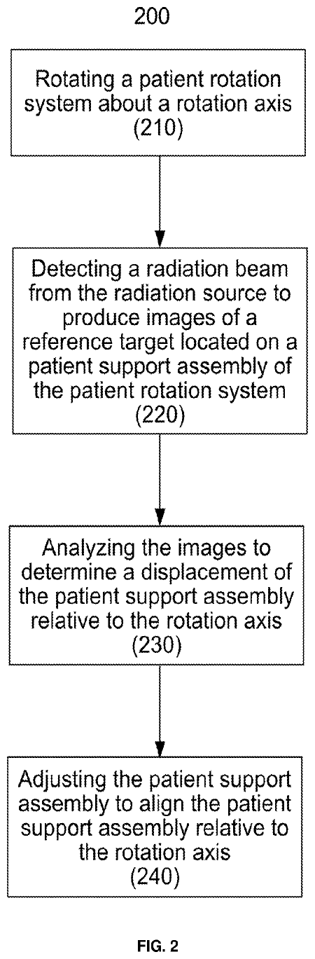

In some embodiments, e.g., as shown in FIG. 2, the technology provides methods 200 for aligning a medical radiation system, e.g., a medical radiation system comprising a patient rotation system and a radiation source (e.g., as shown in FIG. 1A-F). In some embodiments, method 200 comprises rotating 210 the patient rotation system about a rotation axis. In some embodiments, method 200 comprises detecting 220 a radiation beam from the radiation source to produce images of a reference target located on a patient support assembly of the patient rotation system. In some embodiments, the reference target is an embodiment of the reference target 300 as described herein (see, e.g., FIG. 3 and FIG. 4). In some embodiments, method 200 comprises analyzing 230 the images to determine a displacement, or offset, of the patient support assembly relative to the rotation axis. In some embodiments, method 200 comprises adjusting 240 the patient support assembly to align the patient support assembly relative to the rotation axis.

In some embodiments, the method 200 finds use in aligning a medical radiation system, e.g., medical radiation system 100 as shown in FIG. 1. However, the technology is not limited to aligning a medical radiation system 100 as shown in FIG. 1. Accordingly, the technology finds use in aligning any other medical radiation system, such as a radiotherapy system or a medical imaging system.

In some embodiments, the patient rotation system comprises a base structured to rotate. In some embodiments, the base is structured to rotate relative to an axis of the base, such as a vertical axis of symmetry of the base. In some embodiments, the base supports a patient support assembly. In some embodiments, the patient support assembly is adjustably mounted to the base. In some embodiments, adjusting the patient support assembly comprises effectuating a translational movement and/or a rotational movement of the patient support assembly relative to the base. In some embodiments, adjusting the patient support assembly comprises translating and/or rotating one or more of a back rest (e.g., a configurable and movable back rest), a head rest (e.g., a configurable and movable head rest), an arm rest (e.g., a configurable and movable arm rest), a seat member (e.g., a configurable and movable seat member), a shin rest (e.g., a configurable and movable shin rest), and/or a foot brace (e.g., a configurable and movable foot brace).

In some embodiments, the base is fixed at a location (e.g., a floor of a medical provider), e.g., an axis of the base is at a fixed location (e.g., a floor of a medical provider) and the base is structured to rotate relative to the axis and/or the floor. Therefore, embodiments provide that the base comprises an axis and the axis of the base further defines a system axis of rotation with respect to which components of the medical radiation system (e.g., the patient support assembly and the radiation source) are adjusted into alignment. In some embodiments, the components mounted to the patient rotation system (e.g., the patient positioning system, patient positioning apparatus, and/or patient support assembly) are aligned (e.g., centered or zeroed) with respect to the fixed rotation axis. For example, in some embodiments, a horizontal displacement of the patient support assembly with respect to the rotation axis may cause the center of the patient support assembly to precess around the rotation axis rather than rotate on it (e.g., the axis of rotation of the patient support assembly is misaligned such that the axis of rotation of the patient support assembly rotates about a second axis and the patient support assembly precesses about the second axis). Accordingly, embodiments provide that the central axis of the radiation beam is adjusted to align and point towards the isocenter of the patient rotation system.

In some embodiments, e.g., analyzing 230 the images to determine a displacement, or offset, of the patient support assembly relative to the rotation axis comprises comparing a location and/or an orientation of a reference target relative to the rotation axis. In some embodiments, adjusting 240 the patient support assembly to align the patient support assembly relative to the rotation axis comprises aligning a center of rotation (or a center of symmetry) of the reference target with the rotation axis.

In some embodiments, method 200 further comprises analyzing the images to locate a central axis, or isocenter, of the radiation beam relative to the rotation axis. In some embodiments, analyzing the images to locate a central axis, or isocenter, of the radiation beam relative to the rotation axis comprises analyzing the images to determine a displacement of the central axis of the radiation beam relative to the rotation axis (e.g., displacement between the central axis of the radiation beam and the rotation axis). In some embodiments, method 200 further comprises adjusting the radiation source such that the central axis, or isocenter, of the radiation beam intersects the rotation axis. In some embodiments, adjusting the radiation source such that the central axis, or isocenter, of the radiation beam intersects the rotation axis comprises effectuating a translational and/or rotational movement of the radiation source. In some embodiments, adjusting the radiation source further comprises adjusting or varying operational characteristics of the radiation source to change a direction of the radiation beam.

In some embodiments, the medical radiation system comprises a second radiation source. For example, in some embodiments, the medical radiation system comprises a first radiation source that is an imaging radiation source for producing images of a patient during treatment and a second radiation source this a therapeutic or treatment radiation source for treating a patient. In some embodiments, the first radiation source and/or the second radiation source are static (e.g., following adjustment). In some embodiments, the first radiation source and/or the second radiation source is/are fixed in place during normal operation to prevent them from becoming misaligned.

In some embodiments, method 200 further comprises detecting a second radiation beam from a second radiation source, e.g., to produce additional images of the reference target. In some embodiments, method 200 further comprises analyzing the additional images to determine a displacement of the patient support assembly relative to the rotation axis. In some embodiments, method 200 further comprises analyzing the additional images to locate a central axis of the second radiation beam relative to the rotation axis or to determine a displacement of the central axis of the second radiation beam relative to the rotation axis; and adjusting the radiation source such that the central axis of the second radiation beam intersects the rotation axis.

In some embodiments, method 200 further comprises adjusting the first radiation source and/or the second radiation source such that the central axis of the second radiation beam intersects the central axis of the first radiation beam. In some embodiments, the two radiation beams intersect at the rotation axis of the patient rotation system.

In some embodiments, the medical radiation system further comprises an imaging device for detecting the radiation beam and producing images of the reference target (e.g., a detector). In some embodiments, the medical radiation system comprises a plurality of imaging devices, each imaging device being associated with a different radiation source. In some embodiments, each imaging device is located opposite, or effectively or substantially opposite, its associated radiation source relative to the patient support assembly. Therefore, embodiments provide that during execution of method 200, the reference target on the patient support assembly is located between a radiation source and its associated imaging device, such that the radiation beam from the radiation source passes through the reference target before being received by the imaging device to produce images of the reference target.

In some embodiments, the images of the reference target comprise at least two images (e.g., at least a first image and a second image) of the reference target produced for different angles of rotation of the patient rotation system. In some embodiments, the images of the reference target comprise at least four images (e.g., at least a first image, a second image, a third image, and a fourth image) of the reference target produced at orthogonal angles of rotation of the patient rotation system. In some embodiments, analyzing 230 the images to determine a displacement, or offset, of the patient support assembly relative to the rotation axis comprises producing a number of images of the reference target to provide a location of the reference target in three-dimensional space.

Accordingly, in some embodiments methods 200 comprise providing the location of the reference target in three-dimensional space. In some embodiments, the images of the reference target produced by each radiation source are analyzed independently to locate the reference target relative to the rotation axis. In some embodiments, the images of the reference target produced by each radiation source are analyzed in combination to locate the reference target relative to the rotation axis.

In some embodiments, the reference target provides one or more points of reference for determining the position of the patient support assembly or of the radiation beam relative to the rotation axis or any other fixed axis, point, or location. In some embodiments, the reference target is a point or surface on the patient support assembly. In some embodiments, the reference target is a separate device comprising markers of known dimension, composition (e.g., known materials, known Z, and/or known Z_{eff}), and configuration that are attached to the patient support assembly and rotated by the patient rotation system.

In some embodiments, the reference target is attached at a fixed location of the patient support assembly (and/or of the patient rotation system) prior to rotating 210 the patient rotation system about a rotation axis. In some embodiments, the reference target is attached to the center of the patient support assembly. In some embodiments, the patient support assembly is adjusted such that its center is moved into alignment with the rotational axis of the patient rotation system while adjusting 240 the patient support assembly to align the patient support assembly relative to the rotation axis.

In some embodiments, the reference target is attached to the patient support assembly using a quality assurance (QA) interface, indexes, belts, straps, or other fastening components provided on the patient support assembly.

In some embodiments, the reference target is attached to the patient support assembly through an interface. In some embodiments, an interface provides a fixed positioning of the reference target relative to the patient support assembly (and/or relative to the patient rotation system). For example, in some embodiments, the interface reliably positions the target on a center of the patient support assembly, thus allowing a position and/or orientation of the patient support assembly to be determined by using the reference target. In some embodiments, the placement of the interface on the patient support assembly is used to calibrate and/or to define the zero position of the patient support assembly (and/or used to calibrate and/or to define the zero position of the patient rotation system). An offset or poor reproducibility in the placement of the reference target on the patient support assembly may result in a variation in the apparent zero position following execution of method 200. Therefore, in some embodiments, method 200 further comprises attaching, or mounting, the reference target to the patient support assembly. In some embodiments, the patient support assembly comprises an interface for attaching or mounting the reference target at a fixed position on the patient support assembly.

In some embodiments, the method 200 provides a method to minimize the radiation isocenters of a treatment beam and/or an imaging beam (e.g., by directing the central axis of the radiation beam to intersect with the rotation axis). In some embodiments, the method 200 provides a method to align a treatment beam using features in images of edges of a beam shaping system. In some embodiments, a medical radiation system comprises a beam shaping system that attenuates a treatment beam to shape its intensity profile, e.g., to match the profile of a tumor. In some embodiments, methods comprise aligning a beam shaping system with the central axis of the treatment beam, e.g., to ensure that the shape of the radiation field is symmetric around the beam central axis and/or to optimize the shape of the collimator leaves, which are adjustable parts of the beam shaping system.

### Reference Target

In some embodiments, e.g., as shown in FIG. 3 and FIG. 4, the technology relates to a reference target 300 (also known as a “fiducial phantom”). In some embodiments, the reference target 300 finds use in an embodiment of method 200 described herein.

In some embodiments, the reference target 300 comprises a body 310 and a plurality of markers 320 fixed to the body. In some embodiments, the plurality of markers 320 is fixed to the body in a prearranged configuration. In some embodiments, the body 310 is made from a material with a low attenuation coefficient (e.g., a radiotransparent or “radiolucent” material). In some embodiments, the body 310 comprises a homogenous material with a low attenuation coefficient (e.g., a radiotransparent or “radiolucent” material). In some embodiments, the material having a low attenuation coefficient is, e.g., poly(methyl methacrylate), which is also known as acrylic glass and sold commercially under the name PERSPEX. In some embodiments, the body 310 comprises ridges.

In some embodiments, the markers 320 are made from a material with a high attenuation coefficient (e.g., a “radiopaque” material). In some embodiments, the material having a high attenuation coefficient is, e.g., tungsten. Thus, the contrast between body 310 and markers 320 provides for distinguishing the body 310 from the markers 320 when the reference target 300 is imaged using a radiation source. In some embodiments, a first marker of the plurality of markers 320 is made from a first material having a high attenuation coefficient and a second marker of the plurality of markers 320 is made from a second material having a high attenuation coefficient. In some embodiments, the first material is a different material than the second material. Thus, embodiments provide that a first marker of the plurality of markers 320 may be distinguished from a second marker of the plurality of markers 320 when the reference target 300 is imaged using a radiation source. Further embodiments provide that a third, fourth, fifth, sixth, seventh, eighth, ninth, tenth, . . . , and/or nth marker is/are made of a third, fourth, fifth, sixth, seventh, eighth, ninth, tenth, . . . , and/or nth material having a high attenuation coefficient so that one or more markers of the first, second, third, fourth, fifth, sixth, seventh, eighth, ninth, tenth, . . . , and/or nth marker(s) is/are distinguishable from one or more of the other markers of the plurality of markers 320 when the reference target 300 is imaged using a radiation source.

A radiolucent material such as air has a Hounsfield number of approximately—1000 and a radiolucent material such as water has a Hounsfield number of 0 (zero). Body tissues have Hounsfield numbers of, e.g., −900 to −750 (e.g., for lung), −100 to −50 (e.g., for fat), and 500 to 3000 (e.g., for bone with trabecular bone being less dense (e.g., lower Hounsfield number) and cortical bone being denser (e.g., higher Hounsfield number)). Radiopaque markers typically comprise a material having a Hounsfield number in the same range as bone (e.g., 500 to 3000 (e.g., 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000)).

In some embodiments, one or more of the markers 320 has a shape, e.g., to facilitate identification and/or recognition in a radiological image. In some embodiments, one or more of the markers 320 has a shape that is a sphere, ellipsoid, prism, pyramid, elongated bar, or torus. In some embodiments, one or more of the markers 320 has a shape that is a circle, square, rectangle, polygon, or line when projected onto a two-dimensional plane.

In some embodiments, the body 310 is a shape having rotational symmetry (e.g., a cylinder or sphere). In some embodiments, the body 310 has a shape that allows it to be attached (e.g., fixed) to a patient support assembly (e.g., a patient support assembly 120 as shown in FIG. 1) where a patient would be placed.

In some embodiments, e.g., as shown in FIG. 3, the markers 320 are configured in a rotationally asymmetric arrangement, that is, an arrangement that does not repeat itself during one rotational turn of body 310. In this way, each marker is visible at each stage of the rotation process such that it contributes to each image of fiducial phantom 300. In some embodiments, markers 320 are fixed to body 310 in a configuration such that each marker is at least partly exposed or visible along a straight line of sight to the radiation beam at each angle (or at most angles) of rotation of the patient rotation system. In some embodiments, the markers 320 are configured in a symmetrical arrangement.

In some embodiments, the markers 320 are arranged to lie on a plane 330. In some embodiments, the markers 320 are arranged to lie on a plane 330 that is tilted or at an angle relative to the center of rotation 340 of the reference target 300. Accordingly, in some embodiments, the center of rotation 340 is not perpendicular or orthogonal to plane 330.

In some embodiments, the reference target comprises a central marker 322. In some embodiments, the central marker 322 aligns with a center of the patient support assembly (and with center of rotation 340) when the reference target 300 is attached to the patient support assembly. Accordingly, embodiments provide that the non-central, or offset, markers 320 may be used to determine a tilt of fiducial phantom 300. In some embodiments, the tilted arrangement of markers 320 avoids overlap between the markers. e.g., at angles for which detecting the location of two overlapping markers may be difficult.

In some embodiments, the technology provides methods for measuring the tilt of the reference target 300, e.g., at one or more angles. In some embodiments, the technology provides methods for correcting an isocenter size (e.g., an isocenter that is too large due to beam misalignment) by measuring the tilt of the reference target 300 at one or more angles. For example, when the radiation beam is misaligned and the patient support assembly is aligned with the rotation axis, the tilt of the reference target 300 will not change (e.g., will not substantially change, will not effectively change, and/or will not detectably change) as the patient support assembly is rotated but the central marker 322 will appear to move during rotation (e.g., the central marker 322 may appear on the left of the radiation isocenter at a first rotation angle and the central marker 322 may appear on the right of the radiation isocenter at a second rotation angle that is different than the first rotation angle; the greatest extent of movement may be detected between a first rotation angle and a second rotation angle that are 180° apart).

Conversely, when the radiation beam is aligned and the patient support assembly is misaligned with the rotation axis, the location of the central marker 322 will not change (e.g., will not substantially change, will not effectively change, and/or will not detectably change) but the tilt of the non-central markers will vary with rotation angle (e.g., the non-central markers may appear to tilt left at a first rotation angle and the non-central markers may appear to tilt right at a second rotation angle that is different than the first rotation angle; the greatest extent of difference in tilt may be detected between a first rotation angle and a second rotation angle that are 180° apart).

In some embodiments, the reference target 300 finds use in determining the displacement of the radiation beam with respect to a physical device such as a patient rotation system or another quality assurance device. In some embodiments, the reference target 300 finds use in determining the displacement of the radiation beam with respect to a physical device that rotates. In some embodiments, the reference target 300 finds use in determining the displacement of the isocenter of a radiation beam (e.g., a treatment beam or an imaging beam) with respect to a patient rotation system and/or a quality assurance device. In some embodiments, the reference target 300 finds use in verifying that the isocenter size and/or that the coincidence of the isocenters (MV & kV) meet a given specification. In some embodiments, the reference target 300 finds use in measuring an isocenter size. In some embodiments, the reference target 300 finds use in measuring the difference and/or coincidence of the isocenters (MV & kV).

In some embodiments, the reference target 300 locates into a fixed position on the patient rotation system and measuring the location of the markers is used to align the patient rotation system (or equipment mounted on the patient rotation system) to a reference point (e.g., the isocenter). In some embodiments, methods comprising measuring the location of the markers on a reference target 300 that is located at a fixed position on the patient rotation system finds use during initial installation and setup of a radiation system, patient positioning system, patient positioning apparatus, patient rotation system, and/or source. In some embodiments, methods comprising measuring the location of the markers on a reference target 300 that is located at a fixed position on the patient rotation system finds use for maintenance and/or for quality assurance of a radiation system, patient positioning system, patient positioning apparatus, patient rotation system, and/or source.

In some embodiments, the reference target 300 finds use in determining a displacement of the patient support assembly relative to the rotation axis. For example, in some embodiments, determining the displacement of the patient support assembly relative to the rotation axis is used to “zero” the location for one or more of the and x, y, and z axes' positions relative to the rotation axis. In some embodiments, determining the displacement of the patient support assembly relative to the rotation axis is used to “zero” a rotation around one or more of the pitch, roll, and yaw axes of rotation. In some embodiments, the technology provides that determining the displacement of the patient support assembly relative to the rotation axis; “zeroing” the location for one or more of the x, y, and z axes' positions relative to the rotation axis; and/or “zeroing” a rotation around one or more of the pitch, roll, and yaw axes of rotation is/are performed for all movement planes using one set of images, e.g., because the arrangement of the markers 320 provides that at least one marker is always visible and the central marker 320 identifies the center of reference target 300 and of the patient support assembly. In some embodiments, the reference target provides for aligning the central axis of both an imaging radiation source and a treatment radiation source such that the imaging radiation source and the treatment radiation source intersect with the isocenter of the patient rotation system (e.g., the rotation axis). Therefore, reference target 300 finds use in aligning one or more of the central axis of the treatment beam, the central axis of the imaging beam, the rotation axis, and/or the “zero” position of the patient support assembly.

In some embodiments, the arrangement of the markers 320 of the reference target 300 provides for calculating different quantities using the same set of images of the reference target 300. For example, a set of images obtained for a full-cycle rotation of the reference target 300 allows one to calculate: the location of the rotation axis (or isocenter) of the patient rotation system; the distance between the central axis of the treatment beam and the rotation axis; the distance between the central axis of the imaging beam and the rotation axis; the distance between the central axes of the imaging beam and the treatment beam; and the distance between a fixed location on the patient rotation system (as identified by the markers through the interface that mounts reference target 300 to the patient support assembly) and the current readings of the patient rotation system location (e.g., x, y, z axes: pitch, yaw, and roll). In some embodiments, measuring the distance between a fixed location on the patient rotation system (as identified by the markers through the interface that mounts reference target 300 to the patient support assembly) and the current readings of the patient rotation system location (e.g., x, y, z axes; pitch, yaw, and roll) comprising providing an interface that allows the reference target 300 to be mounted to a fixed, reproducible location on the patient support assembly (or the patient rotation system).

### Systems

In some embodiments, the technology provides systems comprising a reference target, e.g., systems comprising a medical radiation system and a reference target (e.g., as shown in FIG. 1A-F). In some embodiments, the technology provides a system comprising a medical radiation system 100 and a reference target 170 (e.g., a reference target 170 comprising a body and a plurality of markers fixed to the body. For example, in some embodiments, the reference target is an embodiment of the reference target 300 as described herein (see, e.g., FIG. 3 and FIG. 4).

In some embodiments, the patient rotation system 110 comprises a patient positioning system (e.g., comprising a patient positioning apparatus or a configurable patient support assembly 120) mounted onto a base 130. Base 130 is structured to rotate about an axis 131 of base 130. In some embodiments, base 130 is structured to rotate about a vertical axis of symmetry of base 130. In some embodiments, the patient positioning system, patient positioning apparatus, and/or configurable patient support 120 is/are as described in U.S. Pat. App. Pub. No. 20200268327 and U.S. Pat. App. Ser. No. 63/237,513, each of which is incorporated herein by reference.

In some embodiments, the patient support assembly 120 is operatively coupled to base 130 such that the patient support assembly 120 rotates together with base 130 (e.g., around axis 131). In some embodiments, the systems comprise a radiation source. For example, some embodiments of systems comprise a first radiation source 150 structured to generate a beam 151 of electromagnetic radiation. In some embodiments, the first radiation source 150 is a kilovoltage (kV) or a megavoltage (MV) x-ray radiation source. First radiation source 150 may be a therapeutic radiation source or an imaging radiation source. In some embodiments, systems further comprise a second radiation source 152 structured to generate a second beam 153 of electromagnetic radiation. Thus, in some embodiments, systems comprise two radiation sources, e.g., a first radiation source that is a therapeutic radiation source and a second radiation source that is an imaging radiation source. In some embodiments, radiation source 150 is a static source, e.g., a source that is not moveable during normal operation (e.g., during radiotherapy). Accordingly, the radiation source 150 may translate, revolve, and/or rotate during a calibration or alignment procedure. In some embodiments, first radiation source 150 is a static source and/or second radiation source 152 is a static source. Accordingly, the first radiation source 150 and/or the second radiation source 152 may translate, revolve, and/or rotate during a calibration or alignment procedure.

Furthermore, embodiments provide that the radiation beam 151 from the first radiation source 150 is perpendicular to the axis of rotation 131 of base 130 (e.g., following the alignment procedure) and/or that the radiation beam 153 from the second radiation source 152 is perpendicular to the axis 131 of rotation of base 130 (e.g., following the alignment procedure). In some embodiments, the radiation source 150 is oriented such that the radiation beam 151 intersects the axis of rotation 131. In some embodiments, an isocenter of the radiation beam intersects the axis of rotation 131.

In some embodiments, radiation source 150 is structured to direct a radiation beam 151 in the direction of patient support assembly 120. Accordingly, when a reference target 170 is positioned on the patient support assembly 120, radiation source 150 is structured to direct a radiation beam 151 in the direction of the reference target 170. In some embodiments, systems comprise a detector 160 (e.g., a detection panel) provided opposite the radiation source 150 to detect the radiation beam 151 that traverses the reference target 170. In some embodiments, detector 160 is an imaging device that produces a signal and/or data for generating an image produced by the radiation beam 151. In some embodiments, an additional (e.g., second) detector 162 is associated with the second radiation source 152.

Embodiments of systems find use in aligning a medical radiation system, e.g., a medical radiation system comprising a patient rotation system and a radiation source (e.g., as shown in FIG. 1A-F). In some embodiments, systems are structured to rotate a patient rotation system about a rotation axis. In some embodiments, systems comprise a radiation source structured to produce a radiation beam that is used to produce images of a reference target located on a patient support assembly of the patient rotation system.

As described herein, in some embodiments, systems comprise a patient rotation system. In some embodiments, the patient rotation system comprises a base structured to rotate. In some embodiments, the base is structured to rotate relative to an axis of the base, such as a vertical axis of symmetry of the base. In some embodiments, the base supports a patient support assembly. In some embodiments, the patient support assembly is adjustably mounted to the base. In some embodiments, systems are structured to adjust the patient support assembly by effectuating a translational movement and/or a rotational movement of the patient support assembly relative to the base. In some embodiments, systems are structured to adjust the patient support assembly by translating and/or rotating one or more of a back rest (e.g., a configurable and movable back rest), a head rest (e.g., a configurable and movable head rest), an arm rest (e.g., a configurable and movable arm rest), a seat member (e.g., a configurable and movable seat member), a shin rest (e.g., a configurable and movable shin rest), and/or a foot brace (e.g., a configurable and movable foot brace).

In some embodiments, the base is fixed at a location (e.g., a floor of a medical provider), e.g., an axis of the base is at a fixed location (e.g., a floor of a medical provider) and the base is structured to rotate relative to the axis and/or the floor. Therefore, embodiments provide that the base comprises an axis and the axis of the base further defines a system axis of rotation with respect to which components of the medical radiation system (e.g., the patient support assembly and the radiation source) are adjusted into alignment (e.g., with a beam). In some embodiments, the components mounted to the patient rotation system (e.g., the patient positioning system, patient positioning apparatus, and/or patient support assembly) are aligned (e.g., centered or zeroed) with respect to the fixed rotation axis. For example, in some embodiments, a horizontal displacement of the patient support assembly with respect to the rotation axis may cause the center of the patient support assembly to precess around the rotation axis rather than rotate on it (e.g., the axis of rotation of the patient support assembly is misaligned such that the axis of rotation of the patient support assembly rotates about a second axis and the patient support assembly precesses about the second axis). Accordingly, embodiments provide that the central axis of the radiation beam is adjusted to align and point towards the isocenter of the patient rotation system.

In some embodiments, systems comprise a software component comprising instructions for rotating a patient rotation system about a rotation axis. In some embodiments, systems comprise a software component comprising instructions for controlling a radiation beam. In some embodiments, systems comprise a software component comprising instructions for receiving a signal and/or data from a detector for producing images. e.g., images of a reference target. In some embodiments, systems comprise a software component comprising instructions for analyzing the images to determine a displacement, or offset, of the patient support assembly relative to the rotation axis. In some embodiments, systems are structured to adjust the patient support assembly to align the patient support assembly relative to the rotation axis. For example, in some embodiments, systems comprise a component (e.g., a motor, linear actuator, adjustment screw, etc.) that is used to adjust the patient support assembly to align the patient support assembly relative to the rotation axis. In some embodiments, the software component comprising instructions for analyzing images to determine a displacement, or offset, of the patient support assembly relative to the rotation axis comprises instructions for comparing a location and/or an orientation of a reference target relative to the rotation axis. In some embodiments, systems are structured to adjust the patient support assembly to align the patient support assembly relative to the rotation axis. For example, in some embodiments, systems comprise a component (e.g., a motor, linear actuator, adjustment screw, etc.) that is used to align a center of rotation (or a center of symmetry) of the reference target with the rotation axis.

In some embodiments, systems comprise a software component comprising instructions for analyzing images to locate a central axis, or isocenter, of a radiation beam relative to the rotation axis. In some embodiments, analyzing the images to locate a central axis, or isocenter, of the radiation beam relative to the rotation axis comprises analyzing the images to determine a displacement of the central axis of the radiation beam relative to the rotation axis (e.g., displacement between the central axis of the radiation beam and the rotation axis). In some embodiments, systems are structured to adjust the radiation source such that the central axis, or isocenter, of the radiation beam intersects the rotation axis. In some embodiments, systems are structured to adjust the radiation source such that the central axis, or isocenter, of the radiation beam intersects the rotation axis by effectuating a translational and/or rotational movement of the radiation source. In some embodiments, systems are structure to adjust the radiation source by adjusting or varying operational characteristics of the radiation source to change a direction of the radiation beam.

In some embodiments, systems comprise a second radiation source. For example, in some embodiments, systems comprise a first radiation source that is an imaging radiation source for producing images of a patient during treatment and a second radiation source that is a therapeutic or treatment radiation source for treating a patient. In some embodiments, the first radiation source and/or the second radiation source are static (e.g., following adjustment). In some embodiments, the first radiation source and/or the second radiation source is/are fixed in place during normal operation to prevent them from becoming misaligned.

In some embodiments, systems comprise a software component comprising instructions for controlling a second radiation beam. In some embodiments, systems comprise a software component comprising instructions for receiving a signal and/or data from a second detector for producing additional images, e.g., additional images of the reference target. In some embodiments, systems comprise a software component comprising instructions for analyzing the additional images to determine a displacement of the patient support assembly relative to the rotation axis. In some embodiments, systems comprise a software component comprising instructions for analyzing the additional images to locate a central axis of the second radiation beam relative to the rotation axis or to determine a displacement of the central axis of the second radiation beam relative to the rotation axis; and systems are structured to adjust the radiation source such that the central axis of the second radiation beam intersects the rotation axis.

In some embodiments, systems comprise a component (e.g., a motor, linear actuator, adjustment screw, etc.) structured to adjust the first radiation source and/or the second radiation source such that the central axis of the second radiation beam intersects the central axis of the first radiation beam. In some embodiments, the two radiation beams intersect at the rotation axis of the patient rotation system.

In some embodiments, systems comprises an imaging device for detecting a radiation beam and producing images of the reference target (e.g., a detector). In some embodiments, systems comprise a plurality of imaging devices, each imaging device being associated with a different radiation source. In some embodiments, each imaging device is located opposite, or effectively or substantially opposite, its associated radiation source relative to the patient support assembly. Therefore, in some embodiments, systems comprise a reference target located on the patient support assembly between a radiation source and its associated imaging device, such that the radiation beam from the radiation source passes through the reference target and is received by the imaging device, which subsequently transmit data and/or signals to the software component to produce images of the reference target.

In some embodiments, systems comprise images of a reference target. In some embodiments, systems comprise a memory component comprising images of a reference target in digital form. In some embodiments, the images of the reference target comprise at least two images (e.g., at least a first image and a second image) of the reference target produced for different angles of rotation of the patient rotation system. In some embodiments, the images of the reference target comprise at least four images (e.g., at least a first image, a second image, a third image, and a fourth image) of the reference target produced at orthogonal angles of rotation of the patient rotation system.

In some embodiments, systems comprise a software component comprising instructions for determining a displacement, or offset, of the patient support assembly relative to the rotation axis by producing a number of images of the reference target that identify a location of the reference target in three-dimensional space. Accordingly, in some embodiments systems comprise a software component comprising instructions for determining the location of the reference target in three-dimensional space. In some embodiments, the images of the reference target produced by each radiation source are analyzed independently by the software component to locate the reference target relative to the rotation axis. In some embodiments, the images of the reference target produced by each radiation source are analyzed in combination by the software component to locate the reference target relative to the rotation axis.

In some embodiments, systems comprise a reference target located to provide one or more points of reference for determining the position of the patient support assembly or of the radiation beam relative to the rotation axis or any other fixed axis, point, or location. In some embodiments, the reference target is a point or surface on the patient support assembly. In some embodiments, the reference target is a separate device comprising markers of known dimension, composition (e.g., known materials, known Z, and/or known Z_{eff}), and configuration that are attached to the patient support assembly and rotated by the patient rotation system.

In some embodiments, systems comprise the reference target attached at a fixed location of the patient support assembly (and/or of the patient rotation system). In some embodiments, the reference target is attached to the center of the patient support assembly. In some embodiments, the patient support assembly is structured to be adjusted such that its center is moved into alignment with the rotational axis of the patient rotation system while adjusting the patient support assembly to align the patient support assembly relative to the rotation axis.

In some embodiments, the patient support assembly comprises a quality assurance (QA) interface, indexes, belts, straps, or other fastening components. In some embodiments, systems comprise the reference target attached to the patient support assembly using a quality assurance (QA) interface, indexes, belts, straps, or other fastening components provided on the patient support assembly.

In some embodiments, the patient support assembly comprises an interface. In some embodiments, the reference target is attached to the patient support assembly using the interface. In some embodiments, the interface provides a fixed positioning of the reference target relative to the patient support assembly (and/or relative to the patient rotation system). For example, in some embodiments, the interface reliably positions the reference target on a center of the patient support assembly, thus allowing a position and/or orientation of the patient support assembly to be determined (e.g., by the system) using the reference target. In some embodiments, the placement of the interface on the patient support assembly is used to calibrate and/or to define the zero position of the patient support assembly (and/or used to calibrate and/or to define the zero position of the patient rotation system). Therefore, in some embodiments, systems comprise the reference target attached or mounted to the patient support assembly. In some embodiments, the patient support assembly comprises an interface at a fixed position on the patient support assembly and the reference target is mounted to the interface.

In some embodiments, the systems comprise a software component comprising instructions for minimizing the radiation isocenters of a treatment beam and/or an imaging beam (e.g., by directing the central axis of the radiation beam to intersect with the rotation axis). In some embodiments, the systems comprise a software component comprising instructions for aligning a treatment beam using features in images of edges of a beam shaping system. In some embodiments, systems comprise a beam shaping system that attenuates a treatment beam to shape its intensity profile, e.g., to match the profile of a tumor. In some embodiments, systems are structured to align a beam shaping system with the central axis of the treatment beam, e.g., to ensure that the shape of the radiation field is symmetric around the beam central axis and/or to optimize the shape of the collimator leaves, which are adjustable parts of the beam shaping system.

Although the disclosure herein refers to certain illustrated embodiments, it is to be understood that these embodiments are presented by way of example and not by way of limitation.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method of aligning a medical radiation system comprising a patient rotation system and a radiation source, the method comprising:
rotating the patient rotation system about a rotation axis;
detecting a radiation beam from the radiation source to produce images of a reference target located on a patient support assembly of the patient rotation system;
analyzing the images to determine a displacement of the patient support assembly relative to the rotation axis; and
adjusting the patient support assembly to align the patient support assembly relative to the rotation axis.

2. The method of claim 1, wherein the displacement is determined by comparing a location and an orientation of the reference target relative to the rotation axis.

3. The method of claim 1 or 2, wherein adjusting the patient support assembly comprises aligning a center of rotation of the reference target with the rotation axis.

4. The method of any one of claims 1 to 3, further comprising:
analyzing the images to locate a central axis of the radiation beam relative to the rotation axis; and
adjusting the radiation source such that the central axis of the radiation beam intersects the rotation axis.

5. The method of any one of claims 1 to 4, wherein the patient rotation system comprises a base configured to rotate, the base supporting the patient support assembly.

6. The method of any one of claims 1 to 5, wherein the patient support assembly is configured for movement with six degrees of freedom relative to the base.

7. The method of claim 5 or 6, wherein adjusting the patient support assembly comprises effectuating a translational movement of the patient support assembly relative to the base.

8. The method of any one of claims 5 to 7, wherein adjusting the patient support assembly comprises effectuating a rotational movement of the patient support assembly relative to the base.

9. The method of any one of claims 5 to 8, wherein rotating the patient rotation system comprises rotating the base, and wherein the rotation axis is an axis of the base.

10. The method of claim 9, wherein the rotation axis is an axis of symmetry of the base.

11. The method of any one of claims 1 to 10, wherein the medical radiation system further comprises an imaging device for detecting the radiation beam and producing the images of the reference target, the imaging device being located opposite the radiation source relative to the patient support assembly.

12. The method of any one of claims 1 to 11, wherein the images of the reference target comprise at least two images of the reference target produced for different angles of rotation of the patient rotation system.

13. The method of any one of claims 1 to 12, wherein the reference target comprises a body and one or more markers fixed to the body in a prearranged configuration.

14. The method of claim 13, wherein the one or more markers are fixed to the body in a prearranged configuration such that each marker is at least partly exposed to the radiation beam at each angle of rotation of the patient rotation system.

15. The method of claim 13 or 14, wherein the one or more markers are disposed on an imaginary plane in the body, and wherein, when the reference target is located on the patient support assembly, the imaginary plane is tilted relative to the rotation axis.

16. The method of any one of claims 13 to 15, wherein the reference target comprises a central marker disposed in the body at the center of rotation of the reference target.

17. The method of any one of claims 13 to 16, wherein the body is substantially radiolucent to the radiation beam.

18. The method of any one of claims 13 to 17, wherein the one or more markers are opaque to the radiation beam.

19. The method of any one of claims 1 to 18, wherein the radiation source is one of an imaging radiation source or a therapeutical radiation source.

20. The method of any one of claims 1 to 19, wherein the medical radiation system further comprises a second radiation source.

21. The method of claim 20, further comprising:
detecting a second radiation beam from the second radiation source to produce additional images of the reference target;
analyzing the additional images to locate a central axis of the second radiation beam relative to the rotation axis; and
adjusting the second radiation source such that the central axis of the second radiation beam intersects the rotation axis.

22. The method of claim 21, further comprising adjusting at least one of the radiation source and the second radiation source such that the central axis of the second radiation beam intersects the central axis of the radiation beam.

23. The method of any one of claims 1 to 22, further comprising attaching the reference target to the patient support assembly.

24. The method of claim 23, wherein the patient support assembly comprises an interface for attaching the reference target at a fixed position on the patient support assembly.

25. A system comprising:
a medical radiation system; and
a reference target comprising a body and one or more markers fixed to the body in a prearranged configuration.

26. The system of claim 25 further comprising a detector.

27. The system of claim 25 further comprising a patient support assembly.

28. The system of claim 27 wherein said patient support assembly comprises an interface structured to accept said reference target.

29. The system of claim 25 structured to rotate said reference target around an axis orthogonal to an axis between a source and a detector.

30. The system of claim 25, wherein said medical radiation system comprises a static source.

31. The system of claim 25 further comprising a software component comprising instructions for rotating a patient rotation system about a rotation axis;
controlling a radiation beam; receiving a signal and/or data from a detector for producing images; analyzing the images to determine a displacement of the patient support assembly relative to the rotation axis; analyzing images to locate an isocenter of a radiation beam relative to the rotation axis; and/or determining a displacement of the central axis of a radiation beam relative to the rotation axis.

32. The system of claim 25 further comprising a component structured to adjust the patient support assembly to align the patient support assembly relative to a rotation axis of the patient support assembly.

33. The system of claim 25 further comprising a component structured to adjust the position of a radiation source and/or a position of a beam produced by said radiation source.
